(19) United States
(12) Patent Application Publication
(10) Pub. No.: US 2025/0231159 A1
(43) Pub. Date: Jul. 17, 2025

(54) MULTI-GAS DIGITAL CARTRIDGE BASED ON METAL OXIDE MEMS SENSOR ARRAY FOR THE DETECTION OF PATTERNS RELATED TO THE AIR COMPOSITION AND RELATED STABILIZATION METHOD

(71) Applicant: AIRGLOSS S.R.L., Roma (RM) (IT)

(72) Inventor: Ciro FORMISANO, Roma (RM) (IT)

(21) Appl. No.: 18/850,989

(22) PCT Filed: Feb. 10, 2023
(86) PCT No.: PCT/IB2023/051200

§ 371 Date: Sep. 25, 2024

(30) Foreign Application Priority Data
Mar. 28, 2022 (IT) 102022000006020

(51) Int. Cl. G01N 33/00

(52) U.S. Cl. CPC G01N 33/0031; G01N 33/0006

(57) ABSTRACT

Multi-gas digital cartridge based on metal oxide MEMS sensor array for the detection of patterns related to the air composition. The sensitive elements of the cartridge are stabilized, by means of an off gassing and controlled passivation technique, in a production period of 72/100 hours instead of the current 4-6 months necessary for natural stabilization. The cartridge, whose operation is ensured by a. process of dynamic scanning and virtualization of sensitive elements by applying a control voltage, once stabilized with the method described here is able to selectively detect total volatile organic compounds (TVOC) with spectrographic profile grouped by main families (alcohols, ethers, ketones, organic acids, aliphatic hydrocarbons, aromatic hydrocarbons, amines, aldehydes, alkenes, halogenated organic compounds, organic sulfur compounds, organic nitrogen compounds), carbon monoxide (CO), nitrogen dioxide (NO₂), formaldehyde (HCHO), ozone (O₃), oxygen (O₂), ammonia (NH₃). sulfur dioxide (SO₂), hydrogen sulfide (H₂S), hydrogen (H₂), hydrofluoric acid (HF), hydrogen cyanide (HCN), hydrochloric acid (HCL), chlorine dioxide (CIO₂), methyl mercaptan (H₄S), bromine (Br₂), thanks to precise surveys obtained from two distinct measurement channels achieved by selective chemical filtration separation and a pattern recognition and extraction process based on principal component analysis.

### FIELD OF THE INVENTION

The invention relates to the field of sensors provided with MEMS technology and having sensitive elements in metal oxide, suitable for analyzing the composition of the air.

### Prior Art

MEMS (Micro electro mechanical systems) technology has established itself on the microelectronics scenario as a disruptive technology capable of bringing the mechanical dimension to the same level as the microelectronic one. Having been able to dramatically change the design paradigms of electronic and computer systems, taking electromechanical functions to the nanometer level previously only implementable with electrotechnical technologies, it has been recognized by all as the most revolutionary technology of the twenty-first century. Furthermore, given the very small size, levels of system integration are made possible which lead the devices to be functionally all-encompassing, i.e. provided with the necessary analog and digital functions that allow integrating in a single device everything needed to interface with the host microcontroller. The evolution of MEMS is so rapid that in the prior art, the micro dimension is moving to the nano dimension, whereby we are already talking of NEMS (Nano electro mechanical systems).

In this scenario, sensors were the first practical application of MEMS technology to see the light, as the most common sensors used in applications, above all automation ones, were so far of an electromechanical nature (for example pressure, movement, acoustic sensors, such as microphones, etc.).

And among these, it has been possible to create environmental sensors (of gas, luminosity, atmospheric pressure, humidity, temperature, etc.) of extremely convenient dimensions and costs. In particular, MEMS gas sensors allow the implementation of very widespread and low-cost environmental monitoring applications. For example, the AS-MLV-P2 is a sensor of gaseous components in MEMS technology made by AMS which finds an important application field in the Internet of Things network technology due to its features of low power consumption (34 mW) and for the long duration (10 years).

However, the main problems of this type of sensors for gas detection and for the analysis of the composition of the air based on MEMS technology in particular remain the low selectivity, as well as the drift of the baseline and the progressive loss of sensitivity which typically occur in the first 6 months of life, making these devices not very usable to provide quantitative information on the concentration of the various contaminants present in the air. For this reason, MEMS sensors based on metal oxide technology are mainly used to provide coarse qualitative indications on the presence of volatile organic compounds (VOC), expressed as a relative or differential value index with respect to the minimum concentration value detected in the previous 24/72 hours. In fact, to overcome the problem of baseline drift, continuous manipulation and correction techniques are used, without which it would be impossible to ensure the correct functioning of the device, renouncing to a static calibration of the same. Furthermore, today's MEMS sensors based on metal oxide technology have severe limitations in the ability to selectively detect volatile substances, making it impossible to identify and measure specific gases, such as methane, ammonia, formaldehyde, nitrogen monoxide, nitric dioxide or ozone. It is also difficult, with these devices, to accurately detect the total value of volatile organic compounds (TVOC) expressed in parts per million, as these sensors are particularly sensitive to hydrogen, which represents, given its poor selectivity, one of the main interferents.

In order to increase the selectivity of the metal oxide MEMS devices, different techniques have been used, ranging from the use of different materials on the sensitive layer to the diversification of the temperatures of the heaters.

For example, the UST (Umwelt Sensor Technik) patented Triplesensor technology uses three different sensitive materials on the same heater, so as to increase the differentiation between easily oxidizable gases (CO), hardly oxidizable gases (CH4) and reducing gases (NO₂, Or₃). However, the various families of organic volatiles (VOC), hydrocarbons and specific contaminants such as formaldehyde remain indistinguishable with this technique. Furthermore, carbon monoxide measurements exhibit very high cross-sensitivity to highly volatile substances such as alcohols, as the diversification of sensitive materials achieves only partial selectivity.

There are also attempts documented in scientific literature to increase the selectivity of metal oxide devices by varying and/or diversifying the temperature of the heaters, but the results obtained are typically only valid in the academic field and of short duration, as the unstable nature of the metal oxide devices frustrates any attempt to characterize them, with widely variable behaviors in the first 6-8 months of life. The same goes for the implementation of pattern extraction and identification algorithms in combination with metal oxide sensor arrays, as the poor repeatability of the measurement over time makes the identification and quantification of substances in the gaseous phase impractical where present in low concentrations, as in the case of air quality parameters, reserving the use of this approach to quality control in the food, chemical, pharmaceutical fields, where the gaseous concentrations involved are 10 or 100 times higher and the result of the analysis is a simplified qualitative parameter such as “good” or “not good”, “yes” or “no”, as a result of the recognition of some specific patterns downstream of a training carried out by the user, as happens for example in the PEN3 (Portable Electronic Nose) device marketed by Airsense.

The object of the present industrial patent application is therefore to propose a multi-gas digital cartridge based on metal oxide MEMS sensor arrays for the detection of patterns related to the composition of the air. The sensitive elements of the cartridge in question are stabilized, by means of an off gassing and controlled passivation technique, in a production period of 72/100 hours instead of the current 4-6 months necessary for natural stabilization. The cartridge is then calibrated and guaranteed to operate for 24 months, with no need for further calibration or baseline drift correction. The single cartridge, whose operation is ensured by a process of dynamic scanning and virtualization of sensitive elements by applying a control voltage, once stabilized with the method described here is able to selectively detect a large number of molecules, such as: total volatile organic compounds (TVOC) with spectrographic profile grouped by main families (alcohols, ethers, ketones, organic acids, aliphatic hydrocarbons, aromatic hydrocarbons, amines, aldehydes, alkenes, halogenated organic compounds, organic sulfur compounds, organic nitrogen compounds), carbon monoxide (CO), nitrogen dioxide (NO₂), formaldehyde (HCHO), ozone (O₃), oxygen (O₂), ammonia (NH₃), sulfur dioxide (SO₂), hydrogen sulfide (H₂S), hydrogen (H₂), hydrofluoric acid (HF), hydrogen cyanide (HCN), hydrochloric acid (HCL), chlorine dioxide (ClO₂), methyl mercaptan (H₄S), bromine (Br₂), thanks to precise surveys obtained from two distinct measurement channels achieved by selective chemical filtration separation and a pattern recognition and extraction process based on principal component analysis.

### DESCRIPTION OF THE INVENTION

The present patent application for industrial invention is intended to describe and claim a device and a method provided with at least a new and alternative solution to the solutions known to date and/or to meet one or more needs perceived in the art and in particular deducible from the above. To accomplish this object, the inventors have developed a sensor for analyzing the composition of the air based on MEMS technology and having sensitive elements in Metal Oxide (Metal Oxide), capable of selectively detecting a large number of gaseous molecules present in the air with high sensitivity and accuracy. The particular features of this sensor are the construction method described below, through which cartridges provided with arrays of sensitive elements with a double measuring chamber are built, and the innovative stabilization method obtained through an advanced process of accelerated aging as well as calibration in a controlled environment, which is able to reduce the production time up to 72-100 hours compared to the 4-6 months necessary in case of aging and stabilization obtained in a natural way.

The multi-gas digital cartridge subject of this industrial patent application is based on an array of sensing elements which can be adjusted in real time to respond partially selectively and with very high precision and repeatability over time to a certain range of substances within a specific chemical group based on specific molecular size and increased propensity for molecular oxidation and/or reduction. The sensitivity of the elements can be varied and adjusted by applying a control voltage capable of modifying the physical parameters of their surface layer, which then make the element more or less sensitive to various volatile compounds. The application of voltage, or “dynamic scanning”, allows to obtain up to 64 different virtual sensitive elements, each of which is capable of giving a specific, partially selective response to a certain range of substances, and these responses, combined, can be used to identify and distinguish the unique “chemical signature” of different chemical compounds or individual substances in the gaseous phase.

To obtain these results, preferably between 3 and 5 metal oxide sensors are positioned inside the cartridge so that they generate at least three traces from two distinct measurement channels: an active measurement trace and a passive measurement trace, both extracted from a first measurement channel, directly exposed to the air to be analyzed, and a reference trace, extracted from a second chemically filtered channel. Both channels are made by placing the sensitive elements downstream of a chamber made with two hydrophobic PTFE membranes which allows gaseous exchange with the environment by double diffusion.

To obtain the chemically filtered channel, at least one of the sensors is placed downstream of a multilayer filter placed between the two PTFE membranes and made with several layers (preferably 6) of fabric impregnated with chemically absorbent material, such as, by way of non-binding example: micronized activated carbon, activated carbon impregnated with potassium iodide, potassium hydroxide, sodium hydroxide or activated carbon mixed with molecular sieves such as aluminosilicates and in particular zeolites of type 3°, 4°, 5°, 10× and 13×. The combination of several overlapping layers of this filtering material allows modulating the passage of gaseous molecules in a selective way, creating a reference channel.

The sensitive elements involved in the measurement through the two distinct channels produce specific and diversified responses. In particular, two sensitive elements belonging to different channels, i.e. filtered and unfiltered, if suitably modulated by means of equal periodic signals, such as for example a sinusoid, a square wave, a ramp, or a step, are able to supply signals that contain uncorrelated, non-redundant information about the composition of the filtered air and unfiltered air, where the filtered channel allows the passage of molecules with low affinity to the composition of the filter material.

By way of example, a filtered channel with fabric impregnated with micronized activated carbon allows the passage of carbon monoxide and hydrogen, while being able to completely block ethyl alcohol and solvents. Discrimination between hydrogen and carbon monoxide is instead achieved by the modulation technique. The information extracted from the two modulated sensors are two or four periodic signals to which the FFT (Fast Fourier Transform) is applied in order to extract the so-called “features”, i.e. the characteristic information of the signal under analysis, i.e. the complex coefficients from A1 to An and from B1 to Bn, where n is the order of the FFT applied to the signal.

The combination of these coefficients can be represented on a histogram graph where each bar of the histogram represents one of the coefficients. The specific shape of a histogram can be associated with a certain chemical element and represents its unique “chemical signature”.

In this context, in order to discriminate between two different substances, the main components of the “chemical signature” are extracted using the PCA algorithm. This result can also be visually represented on a 2D scatter plot, but as the complexity of the compound to be detected increases, visual representation on a three-dimensional plot may be necessary to distinguish all the elements.

In detail, the sensors are modulated by applying a variable voltage across the heater, thus providing for the variation of the temperature of the sensitive layer, or rather for a dynamic shift in the equilibrium of the surface reaction of the chemisorbed oxygen. Consequently, the resistance Rs measured across the sensitive layer will have such a variation as to generate a periodic signal.

The same appropriately transformed periodic voltage is also applied as bias voltage to the resistive divider made to measure the resistance Rs of a second sensitive element driven with a constant voltage, i.e. temperature, thus creating a disturbance to the dynamic equilibrium of the surface reactions. Also in this case the resistance Rs will be a periodic function.

The functions taken into consideration for the extraction of the pattern can be the following:
1) a periodic function Rs1(t), that is, the resistance of the sensitive layer Rs of the first sensitive element measured having applied to the heater a variable voltage V(t)=f(t), wherein the resistance Rs is measured under application of a constant bias voltage to the sensitive layer. This control operation is defined as “dynamic scan with thermal modulation”;
2) a periodic function Rs2(t), that is, the resistance value obtained from the second sensitive element, to which heater a constant voltage V(t)=k is applied, wherein the resistance Rs is measured having applied to the sensitive layer a variable voltage proportional to the voltage V(t) that is controlling the heater of the first sensitive element. This control operation is defined as “controlled bias isotherm”;
3) a periodic function normalized to the isotherm Rn(t)=Rs1(t)/Rs2(t), that is, the ratio between the functions Rs1(t) and Rs2(t).

It should be specified that, in order of preference, the function referred to in point 1 is used, or, if available, the function referred to in point 3, possibly in combination with the function referred to in point 2.

The comparative analysis of the signals obtained and the subsequent extraction of the patterns from both channels allows the extrapolation of information relating to the composition of the air based on the differentiated presence of molecules with selective capacity with respect to:
Total volatile organic compounds (TVOC) with spectrographic profile grouped by main families (alcohols, ethers, ketones, organic acids, aliphatic hydrocarbons, aromatic hydrocarbons, amines, aldehydes, alkenes, halogenated organic compounds, organic sulfur compounds, organic nitrogenous compounds);
carbon monoxide (CO);
nitrogen dioxide (NO₂);
formaldehyde (HCHO),
ozone (O₃);
oxygen (O₂);
ammonia (NH₃);
sulfur dioxide (SO₂);
hydrogen sulfide (H₂S);
hydrogen (H₂);
hydrofluoric acid (HF);
hydrogen cyanide (HCN);
hydrochloric acid (HCL);
chlorine dioxide (ClO₂);
methyl mercaptan (H₄S);
bromine (Br₂).

In particular, in a configuration in which 3 sensitive elements are used on the unfiltered channel and one sensitive element on the filtered channel for a total of 4 sensitive elements, the sensors will be managed as follows:
the sensitive element on a filtered channel and a sensitive element on an unfiltered channel are managed using a “temperature-modulated dynamic scanning”;
one of the sensitive elements on an unfiltered channel is managed according to the modality isotherm with controlled bias;
a further sensitive element is managed in a “static mode”, that is, with the heater at constant temperature and bias with a constant voltage.

The final result, i.e. the concentration of each of the contaminants, is determined using the values provided by the sensors managed in static mode and those managed with controlled bias isotherm mode, which are individually calibrated using the reference gases on two points, i.e. assigning the average Rs value, i.e. the average of the Rs values over the scanning period, at the reference concentration and at the baseline value obtained by exposure to chromatographic air (pure synthetic air, i.e. free of contaminants and CO₂).

These concentration values are suitably corrected by means of a series of coefficients obtained from the dynamic pattern recognition process as previously described, i.e., as a result of processing the functions Rn(t), Rs1(t) and Rs2(t).

In addition to the aforementioned configuration, it is possible to think of a constructive variant which provides for two or more monolithic MEMS sensors suitable for realizing the two distinct measuring chambers. By way of example, in this perspective, a monolithic Mems sensor composed of 4 metal oxide sensitive elements placed downstream of the chemical filter could create a first filtered measurement channel; and another monolithic sensor, or more than one, each composed of 4 sensitive elements in metal oxide exposed to the air, could realize a second unfiltered measuring channel. With this configuration we would have 4 sensitive elements on a filtered channel and 4/8 sensitive elements on an unfiltered channel, where the elements could be diversified as follows: an element with material for oxidizing gases; two elements with material for hardly reducing gases; an element for easily reducing gases.

Due to the passivation caused by siloxanes, aging, micro-cracking of the surface layer and a marked sensitivity to hydrogen, which is independent of the temperature of the sensitive element (common background interferent), the attempt to increase the sensitivity and selectivity of a metal oxide sensor by thermal modulation technique has always shown so far very unstable results. The presence of a reference measurement channel has instead made it possible to obtain reliable and repeatable information over time, as they are differential with respect to common-mode interferents and independent of the evolution and progressive aging of the single sensitive elements.

Another reason for instability typical of metal oxide based sensors is that their sensitivity over time is inversely proportional to the baseline resistance Ra (which is the resistance value in clean air in the absence of chemical contaminants) with respect to the first turn on of the device.

This sensitivity gradually stabilizes after 10/14 days but decreases progressively in the first 4/6 months of the device's life, making it unsuitable for static calibration. For this reason, metal oxide sensors are typically used for differential measurements over a period of 24/48 hours taking as reference the relative minimum point reached in the previous hours; but this makes them generally unreliable, in particular for the absolute concentration measurements of gases such as, for example, volatile organic compounds (TVOC), carbon monoxide, ozone, methane, nitric dioxide, formaldehyde and ammonia, also due to poor selectivity and high instability over time.

Furthermore, the subsequent behavior of the sensor in the stability zone, i.e. after 4/6 months, when an almost stable trend of the baseline and sensitivity has been reached, also strongly depends on the history that occurred in the previous period, i.e. exposure to chemicals to which it was exposed in this phase, by the degree of relative humidity and by the environmental temperature in which such exposure took place.

To overcome this problem, a method has been developed which is able to accelerate the aging of the sensor up to its stability phase in 72/100 hours, instead of the 4/6 months currently required to obtain natural aging. This method also manages to obtain uniformity of performance between the sensors, which remain stable for at least 24 months, as well as repeatability and uniformity of responses between the various production batches.

The technique is implemented thanks to microclimatic chambers in which it is possible to control the temperature with the precision of +/−0.5° C. and the relative humidity with the precision of +/−3% RH; in these chambers it is possible to dispense various gaseous substances obtained by mixing the contents of certified cylinders; the temperature of the micro heaters of the sensor can instead be controlled, in a range between 10° and 450° C., with an accuracy of +/−3° C., and this is especially important in the first hours of operation of the device, as it then determines the behavior downstream of the stabilization.

The possible stages of an accelerated aging and calibration method in a controlled environment are described below; this method, flexible and adaptable to different operational needs, can be used following the precise sequence, or according to a combination of all or some, of the following phases:
A. first turn on and stabilization at low temperature. Sensitive elements are switched on all together at the same time;
B. the elements are kept at a temperature of about 120° C. for about 4 hours. This allows slow activation of the sensitive layer of the sensor;
C. in this step, a gradual increment of the resistance can be observed until a maximum point is reached, which is followed by a gradual reduction. The maximum point indicates the completion of the first step;
   a. if the maximum is not reached, it is necessary to await more time: the procedure cannot be continued unless/until the resistance has reached its maximum;
D. subsequent exposure inside a conditioning chamber to a constant temperature of about 20-22° C. and to a relative humidity of 50% RH with supply of chromatographic air (in which CO₂ and other chemical impurities are totally absent) and promoting the off-gassing and stabilization of the materials in proximity of the sensitive elements;
E. a temperature modulation with sinusoidal cycling for the sensitive elements during about 8-12 hours, with a minimum temperature of 150° C. and a maximum temperature of 400° C., with a cycle lasting about 5 minutes, while supplying a mix of reducing gases having passivating properties or alternatively a mix of air and CO₂ at a 5% ratio, with the temperature being in the range between 30° and 400° C. In the low temperature step chromatographic air is supplied in order to decontaminate the chamber and normalize the sensor surface. In this way a controlled passivation and a stabilization of the sensitivity of the sensitive elements are obtained;
F. the sensors are let for 12 hours at a constant temperature of 320° C. during which chromatographic air is fed with a relative humidity level of 25%;
G. a further stabilization of the sensitive elements and uniformity check of the resistances RS for all sensors;
H. the elements that do not have the required resistance value are individually subjected to the cycle of the above step F) and thereafter they undergo a new measurement until the goal is reached;
I. turning off of the sensors with a desired/correct R value;
J. a possible repetition of step (G) for a period of 6 hours and further check of the value of the resistances RS;
K. the conditioning chamber is brought to a temperature of 60° C. and the sensors are let in the “turned on” state for 2 hours at a temperature of 350° C. in presence of a chromatographic air flow and a relative humidity of 25%. This procedure has the purpose of decontaminating the entire cartridge and in particular the absorbent material of the multilayer chemical filter;
L. the sensors are turned off for 6-12 hours while continuously supplying chromatographic air at 50% relative humidity and at a temperature of 20-22° C.
M. supply of chromatographic air of relative humidity equal to 50%, in order to recondition said sensitive elements and the multilayer chemical filter;
N. the sensors are turned on for 30 minutes at 400° C.;
O. the sensors are turned on for additional 30 minutes at 250° C.;
P. the sensors are let in the “turned on” state in operative conditions and they are stabilized for 6 hours;
Q. calibration is carried out using certified cylinders/tanks by supplying specific gases with required concentrations, according to the configuring and operational range needs of the cartridge undergoing calibration.

Among the possible combinations of the steps described, a simplified and functioning procedure for calibration and accelerated aging of the sensors was tested in particular, involving only steps A), B), C), Ca), D), E), K), P) and Q) of the method in question, for a total duration of about 100 hours.

### DESCRIPTION OF THE FIGURES

The foregoing advantages, as well as other advantages and features of the present invention, will be illustrated with reference to the accompanying drawings, which are to be considered purely illustrative and not limiting or binding to the effects of the present patent application, in which:

FIG. 1 shows the cross-section of a possible multi-gas digital cartridge 100 based on metal oxide Mems sensors for recognizing patterns relating to the composition of the air, where the different construction parts are visible: a PCB 15 on which Mems sensors 10 are installed having sensitive elements 5 in metal oxide, a polymeric chamber 20 divided into a filtering chamber and an exposure chamber, a first membrane in PTFE (polytetrafluoroethylene) 30 and a selective chemical filter 50 preferably with 6 layers, a sealing cover 60 and a second membrane in PTFE 40;

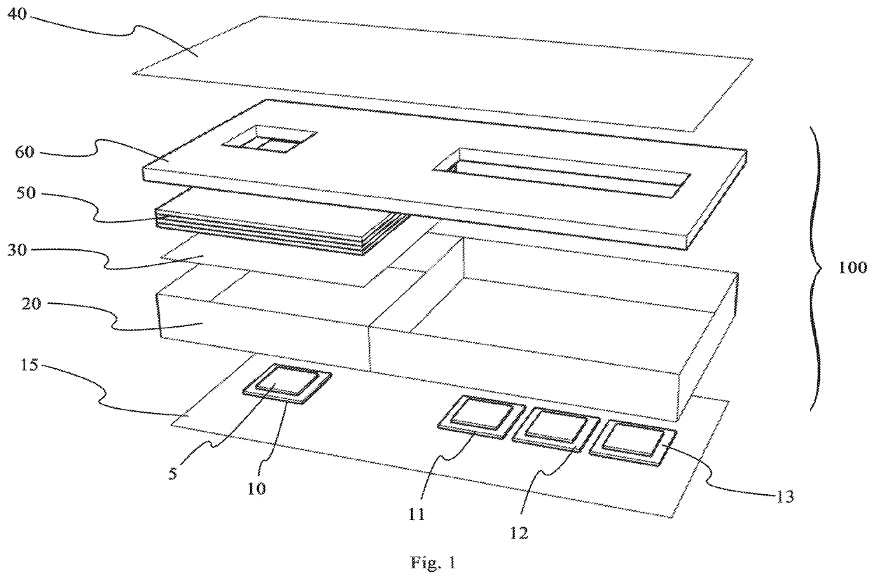

FIG. 2 shows the possible realization of a panel 200 on which ten multi-gas digital cartridges 100 are installed, which will be applied on a machine 300 which is capable of implementing said calibration procedure and accelerated aging of the sensors 10-13.

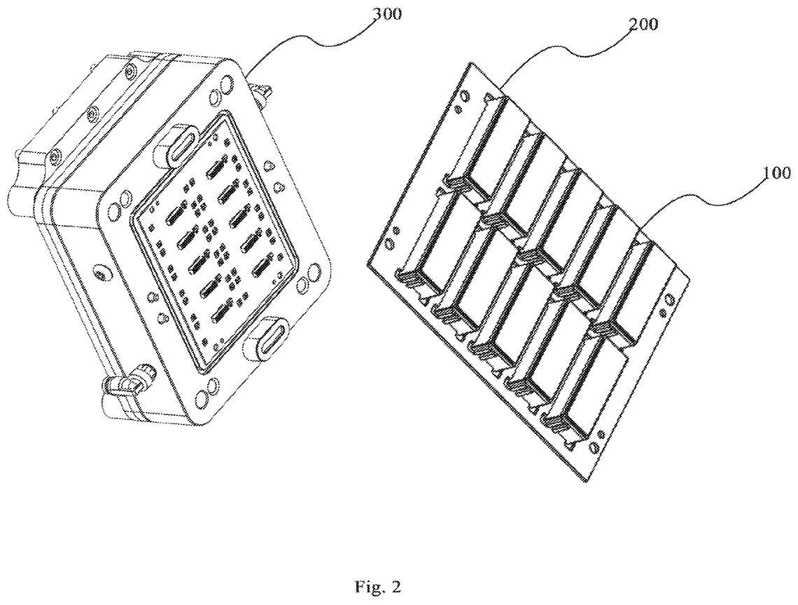

FIG. 3 shows the flowchart for implementing this method in 17 steps.

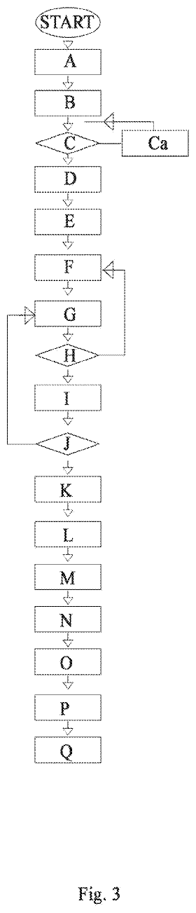

FIG. 4 shows the flow diagram for implementing the method in question reduced for the total duration of 100 hours, consisting of only steps A-E, K and P-Q of the diagram in FIG. 3.

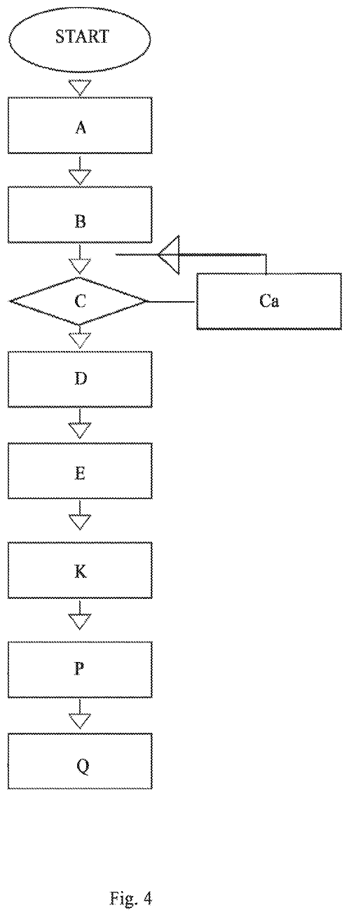

### DETAILED DESCRIPTION OF THE INVENTION

It will be immediately apparent that innumerable variations and modifications (for example relating to shape, dimensions, arrangements and parts with equivalent functionality) may be made to what has been described without departing from the scope of the invention as appears in the appended claims.

The present invention will now be illustrated, purely by way of non-limiting or binding example relating to the present inventive concept, as a multi-gas digital cartridge 100 containing at least a metal oxide sensor 10 located downstream of a selective chemical filter 50, interposed between two membranes in hydrophobic PTFE, inside a first polymeric chamber, and at least three metal oxide sensors 11-13 exposed to the air inside a second polymeric chamber belonging to the same cartridge 100.

The sensitive elements 5, present on the filtered and unfiltered sensors, are modulated through the application of periodic signals, such as for example a sinusoid, a square wave or a step, so that they supply signals at the output which contain uncorrelated information and non-redundant on the composition of the filtered air and unfiltered air, where the filtered channel allows the passage of molecules with low affinity with respect to the composition of the filtering material. This step is able to modify the physical parameters of the elements and therefore to prepare the sensors to respond in a specific and selective way to the gaseous substances; the answers, detected by means of the algorithms and functions described above, can be combined and displayed on typically histogram graphs, scatter graphs, or, depending on the complexity of the measurement, on three-dimensional graphs, which are used to identify and distinguish the “chemical signature” of the different compounds detected.

To implement this project, two measurement chambers of suitable dimensions are created inside a polymeric chamber 20 under which a PCB 15 is inserted having a number of at least four sensors 10, 11, 12 and 13, all provided with a sensitive element 5 based on metal oxide: the first sensor 10 being positioned at the first measurement chamber and the other three sensors 11-13 being positioned at the second one. On the opposite side of said polymeric chamber 20, inside the compartment corresponding to the first sensor 10, a first membrane 30 in PTFE (polytetrafluorethylene) is inserted and subsequently a selective chemical filter 50, preferably with six layers, comprising fabric impregnated with chemically absorbents such as, by way of non-binding example: micronized activated carbon, activated carbon impregnated with potassium iodide, potassium hydroxide, sodium hydroxide or activated carbon mixed with molecular sieves such as aluminosilicates and in particular zeolites of type 3°, 4°, 5th, 10× and 13×. The combination of the various overlapping layers of this filtering material allows modulating the passage of gaseous molecules selectively, creating a channel that will be used for the reference measurements. The three sensors 11-13 exposed to the air at the second compartment inside the polymeric chamber 20 will instead be responsible for the active and passive measurements.

A sealing cover 60 having two suitable openings at the two said compartments is placed as an upper closure of the cartridge 100 and the two openings are further filtered by a second PTFE membrane 40.

The cartridge 100 constructed in this way requires an accelerated stabilization and aging process due to the fact that the sensitive elements 5 undergo the passivation caused by the siloxanes, the micro-fractures of the surface layer and a marked sensitivity to hydrogen.

To fulfill this task, a procedure has been developed, flexible and adaptable to the various operational needs, which can be performed following the sequence, or according to a combination of all or some, of the following steps:
A) first turn on and stabilization at low temperature. Sensitive elements are switched on all together at the same time;
B) the elements are kept at a temperature of about 120° C. for about 4 hours. This allows slow activation of the sensitive layer of the sensor;
C) in this step, a gradual increment of the resistance can be observed until a maximum point is reached, which is followed by a gradual reduction. The maximum point indicates the completion of the first step;
   a. If the maximum is not reached, it is necessary to wait more time: the procedure cannot be continued unless/until the resistance has reached its maximum;
D) subsequent exposure inside a conditioning chamber to a constant temperature of about 20-22° C. and to a relative humidity of 50% RH with supply of chromatographic air (in which CO₂ and other chemical impurities are totally absent) and promoting the off-gassing and stabilization of the materials in proximity of the sensitive elements;
E) a temperature modulation with sinusoidal cycling for the sensitive elements during about 8-12 hours, with a minimum temperature of 150° C. and a maximum temperature of 400° C., with a cycle lasting about 5 minutes, while supplying a mix of reducing gases having passivating properties or alternatively a mix of air and CO₂ at a 5% ratio, with the temperature being in the range between 30° and 400° C. In the low temperature step chromatographic air is supplied in order to decontaminate the chamber and normalize the sensor surface. In this way a controlled passivation and a stabilization of the sensitivity of the sensitive elements are obtained;
F) the sensors are let for 12 hours at a constant temperature of 320° C. during which chromatographic air is fed with a relative humidity level of 25%;
G) a further stabilization of the sensitive elements and uniformity check of the resistances RS for all sensors;
H) the elements that do not have the required resistance value are individually subjected to the cycle of the above step F) and thereafter they undergo a new measurement until the goal is reached;
I) turning off of the sensors with a desired/correct R value;
J) a possible repetition of step (G) for a period of 6 hours and further check of the value of the resistances RS;
K) the conditioning chamber is brought to a temperature of 60° C. and the sensors are let in the “turned on” state for 2 hours at a temperature of 350° C. in presence of a chromatographic air flow and a relative humidity of 25%. This procedure has the purpose of decontaminating the entire cartridge and in particular the absorbent material of the multilayer chemical filter;
L) the sensors are turned off for 6-12 hours while continuously supplying chromatographic air at 50% relative humidity and at a temperature of 20-22° C.
M) supply of chromatographic air of relative humidity equal to 50%, in order to recondition said sensitive elements and the multilayer chemical filter;
N) the sensors are turned on for 30 minutes at 400° C.;
O) the sensors are turned on for additional 30 minutes at 250° C.;
P) the sensors are let in the “turned on” state in operative conditions and they are stabilized for 6 hours;
Q) calibration is carried out using certified cylinders/tanks by supplying specific gases with required concentrations, according to the configuring and operational range needs of the cartridge undergoing calibration.

Among the possible combinations of the steps described, a simplified and functioning procedure for calibration and accelerated aging of the sensors was tested in particular, involving only steps A), B), C), Ca), D), E), K), P) and Q) of the method in question, for a total duration of about 100 hours.

The cartridge 100 thus prepared will be able to detect total volatile organic compounds (TVOC) with a spectrographic profile grouped by main families (alcohols, ethers, ketones, organic acids, aliphatic hydrocarbons, aromatic hydrocarbons, amines, aldehydes, alkenes, halogenated organic compounds, organic sulfur compounds, organic nitrogen compounds), carbon monoxide (CO), nitrogen dioxide (NO₂), formaldehyde (HCHO), ozone (O₃), oxygen (O₂), ammonia (NH₃), sulfur dioxide (SO₂), hydrogen sulfide (H₂S), hydrogen (H₂), hydrofluoric acid (HF), hydrogen cyanide (HCN), hydrochloric acid (HCL), chlorine dioxide (ClO₂), methyl mercaptan (H₄S), bromine (Br₂).

It is clear that modifications, additions or variants may be made to the invention described thus far which are apparent to those skilled in the art, without departing from the scope of protection that is provided by the appended claims.

1. A multi-gas digital cartridge (100) based on metal oxide MEMS sensor arrays, for the detection of patterns related to the air composition, characterized in that it comprises at least two measurement chambers of adequate size created within a polymeric chamber (20) under which a PCB (15) is inserted having a number of at least four Mems sensors (10-13) with respective metal oxide sensitive elements (5), in particular at least a first sensor (10) positioned in the first measurement chamber and at least three additional sensors (11-13) positioned in the second measurement chamber; wherein, on the opposite side of said polymeric chamber (20), inside a compartment facing the first sensor (10), there is a first membrane (30) made of hydrophobic PTFE (polytetrafluoroethylene) and downstream thereof a selective chemical filter (50), preferably including six layers, comprising a fabric impregnated with chemical absorbents, for instance micronized activated carbon, activated carbon impregnated with potassium iodide, potassium hydroxide, sodium hydroxide or activated carbon mixed with molecular sieves like aluminum silicates and in particular zeolites of kind 3A, 4A, 5A, 10× and 13×; wherein a sealing cover (60) with appropriate openings facing said measurement chambers is positioned for closing the upper side of the cartridge (100), and said two openings are further filtered with a second hydrophobic PTFE membrane (40) positioned at the top; wherein, after undergoing the following calibration and stabilization process, described hereinafter, the final thus prepared cartridge (100) is adapted for detecting total organic volatile compounds (TVOC) with spectrographic profile grouped according to the following principal families (alcohols, ethers, ketones, organic acids, aliphatic hydrocarbons, aromatic hydrocarbons, amines, aldehydes, alkenes, halogenated organic compounds, organosulfur compounds, nitrogen organic compounds), carbon monoxide (CO), nitrogen dioxide (NO2), formaldehyde (HCHO), ozone (O3), oxygen (O2), ammoniac (NH3), sulfur dioxide (SO2), hydrogen sulfide (H2S), hydrogen (H2), hydrofluoric acid (HF), hydrogen cyanide (HCN), hydrochloric acid (HCL), chlorine dioxide (ClO2), methyl mercaptan (H4S), and bromine (Br2).

2. A multi-gas digital cartridge (100) based on metal oxide MEMS sensor arrays, for the detection of patterns related to the air composition, according to claim 1, characterized in that said two measurement chambers, which are built within said polymer chamber (20), provide two different measurement channels: the first one faces said first sensor (10) filtered by the selective chemical filter (50) and represents the reference channel; while the second one, which faces said sensors (11-13) exposed to the air to be analyzed represents the channel for carrying out active and passive measurements.

3. A multi-gas digital cartridge (100) based on metal oxide MEMS sensor arrays, for the detection of patterns related to the air composition, according to claim 1, characterized in that the sensitive elements (5) arranged on said sensors (10-13) belonging to different measurement channels are modulated by applying equal periodic signals, like for example a sine wave, a square wave, a ramp or a step, with the purpose of detecting, at the output, signals that contain uncorrelated and non-redundant information on the composition of filtered and unfiltered air; wherein, the functions to be taken into account are—in the order of preference—the function of the following subparagraph A, or if available, the function of the following subparagraph C possibly combined with the function of the following subparagraph B, these functions being:
a periodic function Rs1(t), that is, the resistance of the sensitive layer Rs of the first sensitive element (5) measured having applied to the heater a variable voltage V(t)=f(t), wherein the resistance Rs is measured under application of a constant bias voltage to the sensitive layer; wherein this control operation is defined as “temperature-modulated dynamic scanning”;
a periodic function Rs2(t), that is, the resistance value obtained from the second sensitive element (5), to which heater a constant voltage V(t)=k is applied, wherein the resistance Rs is measured having applied to the sensitive layer a variable voltage proportional to the voltage V(t) that is controlling the heater of the first sensitive element; wherein this control operation is defined as “isotherm with controlled bias”;
a periodic function normalized to the isotherm Rn(t)=Rs1(t)/Rs2(t), that is, the ratio between the functions Rs1(t) and Rs2(t).

4. A multi-gas digital cartridge (100) based on metal oxide MEMS sensor arrays, for the detection of patterns related to the air composition, according to claim 1, characterized in that said sensitive elements (5), arranged on said sensors (10-13), are managed as follows:
the sensitive element (5) on a filtered channel and one among said sensitive elements (5) on an unfiltered channel are managed using a “temperature-modulated dynamic scanning”, as defined in the previous claim;
one among said sensitive elements (5) on an unfiltered channel is managed according to the modality “isotherm with controlled bias” defined in the previous claim;
a further sensitive element (5) is managed in a static mode, that is, with the heater at constant temperature and bias with a constant voltage;
the end result is determined from concentration values which are appropriately corrected using coefficients obtained from the functions Rn(t), Rs1(t) and Rs2(t).

5. A multi-gas digital cartridge (100) based on metal oxide MEMS sensor arrays, for the detection of patterns related to the air composition, according to claim 1, characterized in that the measurements obtained from said two measurement channels, that is, the reference measure detected by said sensor (10) and the active and passive measurements detected by the sensors (11-13) exposed to the air to be analyzed, are read by a dedicated software and are appropriately shown for visual convenience on dispersion graphs in 2D, or depending on the complexity of the measurements on a tridimensional graph, with the purpose of identifying and distinguishing the “chemical signature” of the detected compounds.

6. A method of stabilization and accelerated aging for sensors (10-13) of the MEMS type belonging to a multi-gas digital cartridge (100) according to claim 1, characterized by being carried out using microclimate chambers with controlled temperature and humidity, and into which various gaseous substances can be supplied/introduced from certified cylinders/tanks; wherein said method is flexible and adaptable to different operative needs according to the following sequence, or according to a combination of some or all of the following steps:
A) a first turning on and stabilization at low temperature; all sensitive elements (5) are turned on at the same time;
B) the sensitive elements (5) are maintained at a temperature of about 120° C. for a period of about 4 hours; this allows to slowly activate the sensitive layer of the sensor (10-13);
C) in this step, a gradual increment of the resistance can be observed until a maximum point is reached, which is followed by a gradual reduction; said maximum point indicates the completion of the first step;
C)a. if the maximum is not reached, it is necessary to await more time: the procedure cannot be continued unless/until the resistance has reached its maximum;
D) subsequent exposure inside a conditioning chamber to a constant temperature of about 20-22° C. and to a relative humidity of 50% RH with supply of chromatographic air (in which CO₂ and other chemical impurities are totally absent) and promoting the off-gassing and stabilization of the materials in proximity of the sensitive elements;
E) a temperature modulation with sinusoidal cycling for the sensitive elements during about 8-12 hours, with a minimum temperature of 150° C. and a maximum temperature of 400° C., with a cycle lasting about 5 minutes, while supplying a mix of reducing gases having passivating properties or alternatively a mix of air and CO₂ at a 5% ratio, with the temperature being in the range between 30° and 400° C.; wherein in the low temperature step chromatographic air is supplied in order to decontaminate the chamber and normalize the sensor surface; wherein, in this manner a controlled passivation and a stabilization are achieved for the sensitivity of said sensitive elements;
F) the sensors (10-13) are let for 12 hours at a constant temperature of 320° C. during which chromatographic air is fed with a relative humidity level of 25%;
G) a further stabilization of the sensitive elements (5) and uniformity check of the resistances RS for all sensors (10-13);
H) the elements (5) that do not have the required resistance value are individually subjected to the cycle of the above step F) and thereafter they undergo a new measurement until the goal is reached;
I) turning off of the sensors (10-13) with a desired/correct R value;
J) a possible repetition of step (G) for a period of 6 hours and further check of the value of the resistances RS;
K) the conditioning chamber is brought to a temperature of 60° C. and the sensors (10-13) are let in the “turned on” state for 2 hours at a temperature of 350° C. in presence of a chromatographic air flow and a relative humidity of 25%; this procedure has the purpose of decontaminating the whole cartridge and in particular the absorbing material of the multilayer chemical filter;
L) the sensors (10-13) are turned off for 6-12 hours while continuously supplying chromatographic air at 50% relative humidity and at a temperature of 20-22° C.;
M) supply of chromatographic air of relative humidity equal to 50%, in order to recondition said sensitive elements and the multilayer chemical filter;
N) the sensors (10-13) are turned on for 30 minutes at 400° C.;
O) the sensors (10-13) are turned on for additional 30 minutes at 250° C.;
P) the sensors (10-13) are let in the “turned on” state in operative conditions and they are stabilized for 6 hours;
Q) calibration is carried out using certified cylinders/tanks by supplying specific gases with required concentrations, according to the configuring and operational range needs of the cartridge undergoing calibration.

7. A method of stabilization and accelerated aging for sensors (10-13) of the MEMS type belonging to a multi-gas digital cartridge (100) according to claim 6, characterized in that it comprises only steps A), B), C), Ca), D), E), K), P) and Q), and lasting in total about 100 hours.
